(19) United States
(12) Patent Application Publication
(10) Pub. No.: US 2015/0197487 A1
(43) Pub. Date: Jul. 16, 2015

(54) METHOD FOR PREPARING DIAMINOMALEONITRILE

(71) Applicant: ARKEMA FRANCE, Columbes (FR)

(72) Inventor: Grégory Schmidt, Mornant (FR)

(73) Assignee: ARKEMA FRANCE, Colombes (FR)

(21) Appl. No.: 14/415,003

(22) PCT Filed: Jun. 14, 2013
(86) PCT No.: PCT/FR2013/051395

§ 371 Date: Jan. 15, 2015

(30) Foreign Application Priority Data
Jul. 17, 2012 (FR) 12.56893

(51) Int. Cl. C07C 253/00

(52) U.S. Cl. CPC C07C 253/00

(57) ABSTRACT

A method for manufacturing diaminomaleonitrile, wherein the method includes at least one step during which a cyanohydrin ketone with formula RR′COHCN, R and R′, which are the same or different, being a straight or branched alkyl chain having 1 to 5 carbon atoms, reacts in order to provide diaminomaleonitrile. The reaction step may be carried out in the presence of a catalyst.

### FIELD OF THE INVENTION

The present invention relates to a method for manufacturing diaminomaleonitrile.

### TECHNOLOGICAL BACKGROUND OF THE INVENTION

Diaminomaleonitrile (DAMN) is a known precursor for the synthesis of nitrogen-containing rings such as imidazoles, pyrazines and purines. Recently, however, DAMN has proved to be a precursor for the synthesis of novel lithium salts having interesting properties for improving the performance of Li-ion batteries (WO 2010/023413).

Diaminomaleonitrile is obtained conventionally by polymerization of hydrocyanic acid (HCN) using various types of catalysis.

Document GB1325620 describes the preparation of DAMN by liquid-phase tetramerization of HCN at a temperature between −40° C. and below 0° C. in the presence of a basic catalyst and at least one co-catalyst selected from cyanogen and diiminosuccinonitrile.

We may also cite document U.S. Pat. No. 4,066,683, which describes a method for preparing diaminomaleonitrile by polymerization of HCN in the presence of an alkyl aluminum.

Document U.S. Pat. No. 3,839,406 describes a method for preparing DAMN comprising the following steps: (a) mixing, at room temperature, an alkaline catalyst selected from sodium and potassium cyanide, in a solvent selected from hexamethyl-phosphoramide and hexaethyl-phosphoramide, (b) gradual addition of HCN and stirring the reaction mixture for six hours with an alkali cyanide/HCN molar ratio below 0.25, (c) dilution of the solution with hot water and (d) removal of a blackish amorphous polymer by filtration.

Similarly, document CA1000297 describes a method for manufacturing diaminomaleonitrile by polymerization of HCN in the presence of a compound selected from oxides, hydroxides and cyanides of alkaline-earth metals in a solvent selected from n-methylpyrrolidone, dimethylsulfoxide and dimethylformamide.

In the cases cited above, the starting reactant is hydrocyanic acid, which is a very toxic gas whose use is subject to strict controls, which therefore limits access to DAMN. Now, the increasing market for Li-ion batteries and the interesting properties of lithium salts synthesized from DAMN will lead to high demand for DAMN.

There is therefore a need for a method for preparing diaminomaleonitrile from a starting product that is easy to use, leading to the required product and preferably at a high yield.

### DISCLOSURE OF THE INVENTION

The present invention relates to a method for manufacturing diaminomaleonitrile starting from a ketone cyanohydrin according to reaction 1.

4 RR′C(OH)CN→(CN)₂C═C(NH₂)₂+4 RCOR′ (1)

with R and R′, identical or different, representing a linear or branched alkyl chain having from 1 to 5 carbon atoms.

The method according to the present invention comprising at least one reaction step (1) may be carried out in the presence of a catalyst. The catalyst may be selected from the group consisting of mineral or organic bases whose pK_{A} is between 1 and 14, oxides of alkali metals or alkaline-earth metals and Lewis acids.

As oxides, we may notably mention potassium oxide, sodium oxide or calcium oxide. Silicon oxide may also be suitable.

As Lewis acid, we may notably mention trialkyl aluminum, trialkyl boron or penta-alkyl phosphorus.

Although it is not necessary, the reaction may be carried out in the presence of an aprotic solvent. As solvent, we may mention the nitriles, hexa-alkylphosphoramide, dimethylformamide, dimethylsulfoxide, toluene or xylene.

Additives may also be present in the reaction mixture. As additives, we may notably mention the mercaptans.

The reaction temperature may be between −50 and 200° C., preferably between −20 and 150° C.

The reaction time may vary widely. It is preferably between 1 and 96 hours, advantageously 1 and 72 hours.

According to one embodiment of the method of the present invention, reaction 1 may be carried out in the presence of HCN according to the following scheme.

According to this embodiment, the HCN/ketone cyanohydrin molar ratio y/x may be between 0.001 and 2.5, preferably between 0.005 and 2.

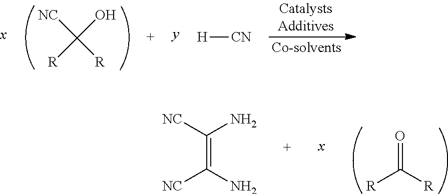

Regardless of the manner of execution, the molar ratio of the amount of catalyst to the total amount of reactants employed (ketone cyanohydrin and optionally HCN)) is preferably between 0.01 and 1, advantageously between 0.05 and 0.95.

The method according to the present invention may further comprise at least one step of recovery of the ketone formed as co-product.

The method may also comprise at least one step of recovery of the catalyst.

The method may also comprise a step of recovery of the excess ketone cyanohydrin.

The method may comprise a step of purification of the diaminomaleonitrile, for example recrystallization or treatment with activated carbon.

The method according to the present invention is easier to carry out with the liquid form of the ketone cyanohydrin. Moreover, this liquid form leads to an improvement in conversion.

Preferably the ketone cyanohydrin is acetone cyanohydrin.

### EXAMPLES

The following examples illustrate but do not limit the invention.

### Example 1

In an autoclave, 5 g of NaCN is added to 65 ml of acetone cyanohydrin. The solution is then stirred at 70° C. for 4 hours. The solution turns brown. The excess acetone cyanohydrin is then neutralized by adding soda. The solution is then filtered and evaporated to give a brown solid (the amount recovered is 16.3 g, corresponding to a yield=85%).

### Example 2

In an autoclave, 10 g of NaCN is added to 130 ml of acetone cyanohydrin. Then 6.54 g of methanethiol is added. The solution is then stirred at 60° C. for 2 hours. The solution turns orange. The excess acetone cyanohydrin is then neutralized by adding soda. The solution is then filtered and evaporated to give an orange-yellow solid (amount recovered=33.7 g corresponding to a yield=88%).

### Example 3

In an autoclave, 28 ml of triethylamine is added to 130 ml of acetone cyanohydrin. The solution is heated to 40° C. and is stirred for 4 hours. The solution turns brown during the reaction. Then 300 ml of water is added and the aqueous phase is extracted with 3×150 ml of ethyl acetate. The organic phases are combined and dried with anhydrous MgSO₄. The solution is then filtered and evaporated. A brown solid is obtained (amount=31.3 g corresponding to a yield=83%).

1. A method for manufacturing diaminomaleonitrile, wherein the method comprises at least one step in which a ketone cyanohydrin of formula RR′COHCN, with R and R′ identical or different, representing a linear or branched alkyl chain having from 1 to 5 carbons, reacts to give diaminomaleonitrile.

2. The method as claimed in claim 1, wherein the reaction step is carried out in the presence of a catalyst.

3. The method as claimed in claim 2, wherein the catalyst is selected from the group consisting of mineral or organic bases whose pKA is between 1 and 14, oxides of alkali metals or alkaline-earth metals and Lewis acids.

4. The method as claimed in claim 3, wherein the Lewis acid is a trialkyl aluminum, trialkyl boron or a penta-alkyl phosphorus.

5. The method as claimed in claim 1, wherein the reaction step is carried out in the presence of an aprotic solvent.

6. The method as claimed in claim 1, wherein the reaction temperature is between −50 and 200° C.

7. The method as claimed in claim 1, wherein the reaction step is carried out in the presence of hydrocyanic acid.

8. The method as claimed in claim 1, wherein the HCN/ketone cyanohydrin molar ratio is between 0.001 and 2.5.

9. The method as claimed in claim 1, wherein the molar ratio of catalyst to the total amount of the reactants is between 0.01 and 1.

10. The method as claimed in claim 1, it wherein the method comprises a step of recovery of the byproduct.

11. The method as claimed in claim 9, wherein the method comprises a step of purification of diaminomaleonitrile.

12. The method as claimed in claim 1, wherein the reaction temperature is between −20 and 150° C.

13. The method as claimed in claim 1, wherein the HCN/ketone cyanohydrin molar ratio is between 0.005 and 2.

14. The method as claimed in claim 1, wherein the molar ratio of catalyst to the total amount of the reactants is between 0.05 and 0.95.
